(19) United States
(12) Patent Application Publication
(10) Pub. No.: US 2015/0328337 A1
(43) Pub. Date: Nov. 19, 2015

(54) PROTECTION FROM OXIDATIVE DAMAGE BY GENE TRANSFER BY GLUTAMATE CYSTEINE LIGASE AND GLUTATHIONE SYNTHASE

(71) Applicant: THE JOHNS HOPKINS UNIVERSITY, Baltimore, MD (US)

(72) Inventor: Peter A. Campochiaro, Baltimore, MD (US)

(21) Appl. No.: 14/652,845

(22) PCT Filed: Dec. 19, 2013
(86) PCT No.: PCT/US2013/076433

§ 371 Date: Jun. 17, 2015

Related U.S. Application Data
(60) Provisional application No. 61/739,542, filed on Dec. 19, 2012.

(51) Int. Cl. A61K 48/00; C12N 9/00; C12N 15/85

(52) U.S. Cl. CPC A61K 48/00; A61K 48/0075; C12N 15/85; C12N 9/93; C12Y 603/02002; C12Y 603/02003

(57) ABSTRACT

An isolated polynucleotide encoding human glutamate cysteine ligase and human glutathione synthase, as well as expression constructs, vectors and pharmaceutical compositions comprising the same, are provided herein. Methods for use of these compositions in the treatment oxidative stress related diseases, including, for example, atherosclerosis, Parkinson's disease, heart failure, myocardial infarction, Alzheimer's disease, diabetes, chronic lung disease, diseases associated with mitochondrial dysfunction, diseases associated with chronic inflammation, retinitis pigmentosa, wet age related macular degeneration, dry age related macular degeneration, diabetic retinopathy, Lebers optic neuropathy, and optic neuritis are also provided.

### REFERENCE TO RELATED APPLICATIONS

This application claims the benefit of U.S. Provisional Patent Application No. 61/739,542, filed on Dec. 19, 2012, which is hereby incorporated by reference for all purposes as if fully set forth herein.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Dec. 17, 2013, is named P12041-02_ST25.txt and is 19,727 bytes in size.

### BACKGROUND OF THE INVENTION

Retinits Pigmentosa (RP) is a type of progressive retinal dystrophy, a group of inherited disorders in which abnormalities of the photoreceptors (rods and cones) or the retinal pigment epithelium (RPE) of the retina lead to progressive visual loss. Affected individuals first experience defective dark adaptation or nyctalopia (night blindness), followed by reduction of the peripheral visual field (known as tunnel vision) and, sometimes, loss of central vision late in the course of the disease. The diagnosis of retinitis pigmentosa relies upon documentation of progressive loss in photoreceptor function by electroretinography (ERG) and visual field testing. The mode of inheritance of RP is determined by family history. At least 35 different genes or loci are known to cause “nonsyndromic RP” (RP that is not the result of another disease or part of a wider syndrome). RP is commonly caused by a mutation in the opsin gene, but can be caused by mutations in a number of other genes expressed systemically or exclusively in the eye. One strategy for therapy is to use gene therapy to replace gene defects, but there are many different mutations in many different genes so that any one such treatment would only apply to a very small percentage of the total patient population with RP. An alternative approach is to identify treatments that do not focus on individual mutations, but instead focus on correcting downstream problems that lead to photoreceptor cell death.

It has been recently found that oxidative stress is a major contributor to cone cell death in RP. After rods die, oxygen utilization in the retina is reduced and the oxygen level in the outer retina is markedly elevated. The present inventors have previously demonstrated that the increased tissue oxygen that occurs as rods die generates reactive oxygen species (ROS) through upregulation of NADPH oxidase as well as mismatches in the electron transport chain. This results in progressive oxidative and nitrosative damage to cones, which are major contributors to cone cell death. Various antioxidants slow cone cell death in multiple models of RP, indicating that antioxidant therapies may provide some benefit to patients with RP regardless of the pathogenic mutation. When free radicals are generated they interact with the first available acceptor they contact, and for antioxidants to prevent damage to critical molecules they must be present in sufficiently high concentrations in correct cellular compartments to reduce chance meetings of radicals with those molecules. This is a difficult requirement for exogenous antioxidants that must penetrate into all cellular compartments and maintain high levels at all times. Bolstering the endogenous antioxidant defense system by ocular gene transfer may provide a more efficient approach that could be used in a complimentary fashion.

Superoxide dismutase 1 (SOD 1) is an important component of the antioxidant defense system in the retina, because mice deficient in SOD1 show increased susceptibility to retinal damage from oxidative stress. In contrast SOD3 knockout mice have no increased susceptibility to retinal damage from oxidative stress. Although one cannot readily perform similar experiments for SOD2 because knockout of SOD2 is embryonic lethal, it was previously thought it would be reasonable to investigate increased expression of SOD2 as well as SOD1 in photoreceptors in the rd1 model of RP. Contrary to the initial hypothesis, it was found that increased expression of SOD1 or SOD2 in the photoreceptors of rd1 mice increased oxidative stress and accelerated cone degeneration (WO2010/005533), which is incorporated by reference herein. It was postulated that although the increase in SOD1 or SOD2 might protect against superoxide radicals, it could lead to an excess of H₂O₂ that overwhelmed endogenous H₂O₂-detoxifying enzymes. Over-expression of members of the glutathione (Gpx) family of H₂O₂-detoxifying enzymes reduced susceptibility to severe oxidative stress in the retina with Gpx4 particularly helpful. Therefore, the tet-on inducible promoter system was used to generate transgenic mice with inducible expression of SOD1, SOD2, cytosolic Gpx4, or catalase targeted to mitochondria (mCatalase). By an elaborate mating scheme, rd10^{+/+} mice were generated with inducible expression of these enzymes alone or in various combinations. Rd10^{+/-} mice with expression of SOD1 or SOD2 alone showed increased oxidative stress and accelerated degeneration of cones, but those that expressed SOD1 and Gpx4 or SOD2 and mCatalase had marked preservation of cone survival and function.

Current treatment of diseases in which oxidative damage plays a major role, including those resulting in retinal degeneration, involves administration of oral antioxidants. This approach is inefficient because the antioxidants must penetrate through the blood-retinal barrier and into retinal cells and be present at all times. As a result, these treatments slow oxidative damage, but do not prevent it.

Therefore, there still exists an unmet need to develop further methods of prevention and treatment of diseases involving oxidative damage, including treatment of diseases of the retina.

### SUMMARY OF THE INVENTION

RP is a blinding disorder for which there are no effective treatments. There is strong evidence indicating that oxidative damage plays a major role in cone cell death in RP, indicating that oxidative damage is a therapeutic target that applies to all patients with RP. In accordance with one or more embodiments, the present invention provides therapeutic methods that are broadly applicable in patients with RP and do not apply only to a small subgroup. Once rods die, the cones are under constant assault from ROS for the remainder of the patient's life.

In accordance with one or more embodiments, the present invention provides compositions and methods for gene transfer of components of the oxidative defense system to provide long term protection for this chronic problem. Embodiments of the present invention provide for the expression of enzymes that detoxify ROS. The compositions and methods of the present invention are highly significant because they establish novel ways to achieve the same goal.

GSH is the major small molecule antioxidant in cells and is able to diffuse into and protect all cellular compartments; thus gene transfer of the two enzymes responsible for synthesis of GSH provides protection throughout the cell. In addition, oxidative damage has been implicated in age-related macular degeneration and thus, in one or more embodiments, the compositions and methods of the present invention will also be useful in that highly prevalent disease which further adds to the significance of the proposal. In addition, there are many neurodegenerative disorders such as Alzheimer's disease, Parkinson's disease, and Amyotrophic Lateral Sclerosis in which oxidative damage plays an important role and will be benefitted by these inventions.

In accordance with an embodiment, the present invention provides an isolated polynucleotide encoding human glutamate cysteine ligase and human glutathione synthase.

In accordance with another embodiment, the present invention provides an isolated polynucleotide encoding human glutamate cysteine ligase and human glutathione synthase and comprises the nucleotide sequences of SEQ ID NOS: 1-3.

In accordance with a further embodiment, the present invention provides an isolated polynucleotide which encodes the nucleotide sequences complementary to the polynucleotide comprising the nucleotide sequences of SEQ ID NOS: 1-3.

In accordance with yet another embodiment the present invention provides a composition comprising the polynucleotide comprising the nucleotide sequences of SEQ ID NOS: 1-3, and a pharmaceutically acceptable carrier.

In accordance with an embodiment, the present invention provides a composition comprising the polynucleotide comprising the nucleotide sequences of SEQ ID NOS: 1-3, and a pharmaceutically acceptable carrier, wherein the composition comprises an expression construct for expression of human glutamate cysteine ligase and human glutathione synthase.

In accordance with an embodiment, the present invention provides a method for the prevention, amelioration, or treatment of a disease or condition associated with oxidative stress in a subject comprising administration of a therapeutically effective amount of any of the compositions described herein, to increase the amount of glutathione expressed in the tissues of the subject.

In accordance with an embodiment, the present invention provides a method for the prevention, amelioration, or treatment of RP in a subject comprising administration of a therapeutically effective amount of any of the compositions described herein, to increase the amount of glutathione expressed in the eye of the subject.

### DETAILED DESCRIPTION OF THE INVENTION

Glutathione (GSH) is a tripeptide, c-L-glutamyl-L-cysteinyl-glycine, found in all mammalian tissues. It has several important functions including detoxification of electrophiles, scavenging ROS, maintaining the thiol status of proteins, and regeneration of the reduced forms of vitamins C and E. GSH is the dominant non-protein thiol in mammalian cells; as such it is essential in maintaining the intracellular redox balance and the essential thiol status of proteins. Also, it is necessary for the function of some antioxidant enzymes such as the glutathione peroxidases.

Intracellular GSH levels are determined by the balance between production and loss. Production results from de novo synthesis and regeneration of GSH from GSSG by GSSG reductase. Generally there is sufficient capacity in the GSSG reductase system to maintain all intracellular GSH in the reduced state, so little can be gained by ramping up that pathway. The major source of loss of intracellular GSH is transport out of cells. Intracellular GSH levels range from 1-8 mM while extracellular levels are only a few μM; this large concentration gradient essentially precludes transport of GSH into cells and once it is transported out of cells, it is rapidly degraded by y-glutamyltranspeptidase. Inhibition of GSH transporters could theoretically increase intracellular GSH levels, but is potentially problematic because the transporters are not specific for GSH and their suppression could lead imbalance of other amino acids and peptides. Thus, intracellular GSH levels are modulated primarily by changes in synthesis.

GSH is synthesized in the cytosol of virtually all cells by two ATP-requiring enzymatic steps: L-glutamate+L-cysteine+ATP→γ-glutamyl-L-cysteine+ADP+Pi and γ-glutamyl-L-cysteine+L-glycine+ATP→GSH+ADP+Pi. The first reaction is rate-limiting and is catalyzed by glutamate cysteine ligase (GCL, EC 6.3.2.2). GCL is composed of a 73 Kd heavy catalytic subunit (GCLC) and a 30 Kd modifier subunit (GCLM) which are encoded by different genes. GCCL is regulated by nonallosteric competitive inhibition by GSH (Ki=2.3 mM) and by the availability of L-cysteine. The apparent Km of GLC for glutamate is 1.8 mM and intracellular glutamate concentration is roughly 10-fold higher so that glutamate is not limiting, but the Km for cysteine is 0.1-0.3 mM which approximates its intracellular concentration. The second reaction is catalyzed by GSH synthase (GS, EC 6.3.2.3) which is 118 Kd and composed of two identical subunits. While GS is not felt to be important in regulation of GSH synthesis under normal conditions, it may play a role under stressful conditions because in response to surgical trauma, GSH levels and GS activity were reduced while GCL activity was unchanged. Furthermore, compared to increased expression of GCLC alone, increased expression of both GCLC and GS resulted in higher levels of GSH. In order to maximize the effects of increasing synthetic enzymes, it is necessary to provide increased levels of cysteine. In cultured neurons, 90% of cysteine uptake occurs through by the sodium-dependent excitatory amino acid transporter (EAAT) system. There are five EAATs and cysteine uptake by neurons occurs predominantly by EAAT3 more commonly known as excitatory amino acid carrier-1 (EAAC1). Under normal circumstances most EAAC1 is in the ER and only translocates to the plasma membrane when activated. This translocation is negatively regulated by glutamate transporter associated protein 3-18 (GTRAP3-18) and suppression of GTRAP3-18 increases GSH levels in neurons. Thus, internalization of cysteine provides a road block for GSH synthesis, but fortunately it can be bypassed by N-acetylcysteine (NAC) which readily enters cells even in the absence of activated EAAC1. Systemically administered NAC gains access to the CNS, increases GSH levels, and provides benefit in neurodegenerative disorders in which oxidative stress is an important part of the pathogenesis. The present inventors have demonstrated that orally administered NAC promotes long term survival of cones in a model of RP.

All cellular compartments must be protected against oxidative damage, including the cytoplasm, mitochondria and the nucleus. The present inventors have previously performed gene transfer of enzymes that detoxify reactive oxygen species, but that approach requires expression of two enzymes in the cytoplasm and two enzymes in mitochondria. In contrast, the present invention provides for protection of all cellular compartments with expression of only two enzymes in the cytosol because GSH is able to diffuse everywhere throughout cells.

In accordance with an embodiment, the present invention provides an isolated polynucleotide encoding human glutamate cysteine ligase and human glutathione synthase.

In accordance with another embodiment, the present invention provides an isolated polynucleotide encoding human glutamate cysteine ligase and human glutathione synthase and has the nucleotide sequences of SEQ ID NOS: 1-3.

Homo sapiens glutamate-cysteine ligase, modifier subunit (GCLM), mRNA:

```
(SEQ ID NO: 1)
    1 atgggcaccg acagccgcgc ggccaaggcg ctcctggcgc gggcccgcac cctgcacctg
  61 cagacgggga acctgctgaa ctggggccgc ctgcggaaga agtgcccgtc cacgcacagc
121 gaggagcttc atgattgtat ccaaaaaacc ttgaatgaat ggagttccca aatcaaccca
181 gatttggtca gggagtttcc agatgtcttg gaatgcactg tatctcatgc agtagaaaag
241 ataaatcctg atgaaagaga agaaatgaaa gtttctgcaa aactgttcat tgtagaatca
301 aactcttcat catcaactag aagtgcagtt gacatggcct gttcagtcct tggagttgca
361 cagctggatt ctgtgatcat tgcttcacct cctattgaag atggagttaa tctttccttg
421 gagcatttac agccttactg ggaggaatta gaaaacttag ttcagagcaa aaagattgtt
481 gccataggta cctctgatct agacaaaaca cagttggaac agctgtatca gtgggcacag
541 gtaaaaccaa atagtaacca agttaatctt gcctcctgct gtgtgatgcc accagatttg
601 actgcatttg ctaaacaatt tgacatacag ctgttgactc acaatgatcc aaaagaactg
661 ctttctgaag caagtttcca agaagctctt caggaaagca ttcctgacat tcaagcgcac
721 gagtgggtgc cgctgtggct actgcggtat tcggtcattg tgaaaagtag aggaattatc
781 aaatcaaaag gctacatttt acaagctaaa agaaggggtt cttaa.
```

Homo sapiens glutamate-cysteine ligase, catalytic subunit (GCLC), transcript variant 1, mRNA:

```
(SEQ ID NO: 2)
      1 atggggctgc tgtcccaggg ctcgccgctg agctgggagg aaaccaagcg ccatgccgac
    61 cacgtgcggc ggcacgggat cctccagttc ctgcacatct accacgccgt caaggaccgg
  121 cacaaggacg ttctcaagtg gggcgatgag gtggaataca tgttggtatc ttttgatcat
  181 gaaaataaaa aagtccggtt ggtcctgtct ggggagaaag ttcttgaaac tctgcaagag
  241 aagggggaaa ggacaaaccc aaaccatcct accctttgga gaccagagta tgggagttac
  301 atgattgaag ggacaccagg acagccctac ggaggaacaa tgtccgagtt caatacagtt
  361 gaggccaaca tgcgaaaacg ccggaaggag gctacttcta tattagaaga aaatcaggct
  421 ctttgcacaa taacttcatt tcccagatta ggctgtcctg ggttcacact gcccgaggtc
  481 aaacccaacc cagtggaagg aggagcttcc aagtccctct tctttccaga tgaagcaata
  541 aacaagcacc ctcgcttcag taccttaaca agaaatatcc gacataggag aggagaaaag
  601 gttgtcatca atgtaccaat atttaaggac aagaatacac catctccatt tatagaaaca
  661 tttactgagg atgatgaagc ttcaagggct tctaagccgg atcatattta catggatgcc
  721 atgggatttg gaatgggcaa ttgctgtctc caggtgacat tccaagcctg cagtatatct
  781 gaggccagat acctttatga tcagttggct actatctgtc caattgttat ggctttgagt
  841 gctgcatctc ccttttaccg aggctatgtg tcagacattg attgtcgctg gggagtgatt
  901 tctgcatctg tagatgatag aactcgggag gagcgaggac tggagccatt gaagaacaat
  961 aactatagga tcagtaaatc ccgatatgac tcaatagaca gctatttatc taagtgtggt
1021 gagaaatata atgacatcga cttgacgata gataaagaga tctacgaaca gctgttgcag
1081 gaaggcattg atcatctcct ggcccagcat gttgctcatc tctttattag agacccactg
1141 acactgtttg aagagaaaat acacctggat gatgctaatg agtctgacca ttttgagaat
1201 attcagtcca caaattggca gacaatgaga tttaagcccc ctcctccaaa ctcagacatt
1261 ggatggagag tagaatttcg acccatggag gtgcaattaa cagactttga gaactctgcc
1321 tatgtggtgt ttgtggtact gctcaccaga gtgatccttt cctacaaatt ggattttctc
1381 attccactgt caaaggttga tgagaacatg aaggtagcac agaaaagaga tgctgtcttg
1441 cagggaatgt tttatttcag gaaagatatt tgcaaaggtg gcaatgcagt ggtggatggt
1501 tgtggcaagg cccagaacag cacggagctc gctgcagagg agtacaccct catgagcata
1561 gacaccatca tcaatgggaa ggaaggtgtg tttcctggac tgatcccaat tctgaactct
1621 taccttgaaa acatggaagt ggatgtggac accagatgta gtattctgaa ctacctaaag
1681 ctaattaaga agagagcatc tggagaacta atgacagttg ccagatggat gagggagttt
1741 atcgcaaacc atcctgacta caagcaagac agtgtcataa ctgatgaaat gaattatagc
1801 cttattttga agtgtaacca aattgcaaat gaattatgtg aatgcccaga gttacttgga
1861 tcagcattta ggaaagtaaa atatagtgga agtaaaactg actcatccaa ctag.
```

Homo sapiens glutathione synthetase (GSS), mRNA:

```
(SEQ ID NO: 3)
      1 atggccacca actgggggag cctcttgcag gataaacagc agctagagga gctggcacgg
    61 caggccgtgg accgggccct ggctgaggga gtattgctga ggacctcaca ggagcccact
  121 tcctcggagg tggtgagcta tgccccattc acgctcttcc cctcactggt ccccagtgcc
  181 ctgctggagc aagcctatgc tgtgcagatg gacttcaacc tgctagtgga tgctgtcagc
  241 cagaacgctg ccttcctgga gcaaactctt tccagcacca tcaaacagga tgactttacc
  301 gctcgtctct ttgacatcca caagcaagtc ctaaaagagg gcattgccca gactgtgttc
  361 ctgggcctga atcgctcaga ctacatgttc cagcgcagcg cagatggctc cccagccctg
  421 aaacagatcg aaatcaacac catctctgcc agctttgggg gcctggcctc ccggacccca
  481 gctgtgcacc gacatgttct cagtgtcctg agtaagacca aagaagctgg caagatcctc
  541 tctaataatc ccagcaaggg actggccctg ggaattgcca aagcctggga gctctacggc
  601 tcacccaatg ctctggtgct actgattgct caagagaagg aaagaaacat atttgaccag
  661 cgtgccatag agaatgagct actggccagg aacatccatg tgatccgacg aacatttgaa
  721 gatatctctg aaaaggggtc tctggaccaa gaccgaaggc tgtttgtgga tggccaggaa
  781 attgctgtgg tttacttccg ggatggctac atgcctcgtc agtacagtct acagaattgg
  841 gaagcacgtc tactgctgga gaggtcacat gctgccaagt gcccagacat tgccacccag
  901 ctggctggga ctaagaaggt gcagcaggag ctaagcaggc cgggcatgct ggagatgttg
  961 ctccctggcc agcctgaggc tgtggcccgc ctccgcgcca cctttgctgg cctctactca
1021 ctggatgtgg gtgaagaagg ggaccaggcc atcgccgagg cccttgctgc ccctagccgg
1081 tttgtgctaa agccccagag agagggtgga ggtaacaacc tatatgggga ggaaatggta
1141 caggccctga aacagctgaa ggacagtgag gagagggcct cctacatcct catggagaag
1201 atcgaacctg agccttttga gaattgcctg ctacggcctg gcagccctgc ccgagtggtc
1261 cagtgcattt cagagctggg catctttggg gtctatgtca ggcaggaaaa gacactcgtg
1321 atgaacaagc acgtggggca tctacttcga accaaagcca tcgagcatgc agatggtggt
1381 gtggcagcgg gagtggcagt cctggacaac ccataccctg tgtga.
```

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

In an embodiment, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art. See, for example, Sambrook et al. (eds.), Molecular Cloning, A Laboratory Manual, 3^{rd} Edition, Cold Spring Harbor Laboratory Press, New York (2001) and Ausubel et al., Current Protocols in Molecular Biology, Greene Publishing Associates and John Wiley & Sons, NY (1994). For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleic acid can comprise any nucleotide sequence that encodes any of the GCLM, GCLC and GSS polypeptides, or proteins, or fragments or functional portions or functional variants thereof For example, the nucleic acid can comprise a nucleotide sequence comprising any of SEQ ID NOs: 1-3, or alternatively can comprise a nucleotide sequence that is degenerate to any of SEQ ID NOs: 1-3.

The invention also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein. In an embodiment, the present invention provides a nucleic acid molecule which is complementary to the full length nucleotide sequence of any of SEQ ID NOs: 1-3.

The invention also provides an isolated or purified polypeptides or proteins or functional portions thereof which are encoded by SEQ ID NOs: 1-3, and are, in an embodiment, provided as SEQ ID NOS: 12-14.

As defined herein, a functional portion or functional variant of any of the GCLM, GCLC and GSS polypeptides, or proteins, or fragments or functional portions or functional variants thereof, includes the catalytic portions of the enzymes.

In an embodiment, the isolated nucleic acid molecule comprises a nucleotide sequence which is substantially the same as, e.g., has at least 50%, e.g., 60%, 70%, 80% or 90% or more, contiguous nucleic acid sequence identity to, one of SEQ ID NOs: 1-3, or the complement thereof.

The nucleic acids of the invention can be incorporated into a recombinant expression vector or expression construct. In this regard, the invention provides recombinant expression vectors comprising any of the nucleic acids of the invention. For purposes herein, the term “recombinant expression vector or expression construct” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages do not hinder the transcription or replication of the vector.

The recombinant expression vector of the invention can be any suitable recombinant expression vector, and can be used to transform or transfect any suitable host. Suitable vectors include those designed for propagation and expansion or for expression or both, such as plasmids and viruses.

The recombinant expression vectors of the invention can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColE1, 2μ plasmid, λ, SV40, bovine papilloma virus, and the like. In one or more embodiments, the vectors used are preferably lentiviral vectors or AAV vectors.

In one or more embodiments, the “expression vector or construct” as used herein is understood as a nucleic acid sequence including a sequence for expression as a polypeptide or nucleic acid (e.g., siRNA, shRNA) operably linked to a promoter and other essential regulatory sequences to allow for the expression of the polypeptide in at least one cell type. In a preferred embodiment, the promoter and other regulatory sequences are selected based on the cell type in which the expression construct is to be used. Selection of promoter and other regulatory sequences for protein expression are well known to those of skill in the art. An expression construction preferably also includes sequences to allow for the replication of the expression construct, e.g., plasmid sequences, virus sequences, etc. For example, expression constructs can be incorporated into replication competent or replication deficient viral vectors including, but not limited to, adenoviral (Ad) vectors, adeno-associated viral (AAV) vectors of all serotypes, self-complementary AAV vectors, and self-complementary AAV vectors with hybrid serotypes, self-complementary AAV vectors with hybrid serotypes and altered amino acid sequences in the capsid that provide enhanced transduction efficiency, lentiviral vectors, or plasmids for bacterial expression.

Desirably, the recombinant expression vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA or RNA based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the GCLM, GCLC and GSS polypeptides, or proteins (including functional portions and functional variants thereof).

The invention further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like. In one or more embodiments, a preferred host cell or population of cells includes the cells of the retina and of the eye.

Also provided by the invention is a population of cells comprising at least one host cell described herein. The population of cells can be a heterogeneous population comprising the host cell comprising any of the recombinant expression vectors described, in addition to at least one other cell, e.g., a host cell (e.g., a skin cell), which does not comprise any of the recombinant expression vectors, or a cell other than a skin cell, e.g., a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

In accordance with an embodiment, the present invention provides a method for the prevention, amelioration, or treatment of a disease or condition associated with oxidative stress in a subject comprising administration of a therapeutically effective amount of any of the compositions described herein, to increase the amount of glutathione expressed in the tissues of the subject.

As used herein, “active oxygen species” or “reactive oxygen species” are understood as understood as transfer of one or two electrons produces superoxide, an anion with the form O₂″, or peroxide anions, having the formula of O₂^{2″} or compounds containing an O—O single bond, for example hydrogen peroxides and lipid peroxides. Such superoxides and peroxides are highly reactive and can cause damage to cellular components including proteins, nucleic acids, and lipids.

An “agent” is understood herein to include a therapeutically active compound or a potentially therapeutic active compound, e.g., an antioxidant. An agent can be a previously known or unknown compound. As used herein, an agent is typically a non-cell based compound, however, an agent can include a biological therapeutic agent, e.g., peptide or nucleic acid therapeutic, e.g., siRNA, shRNA, cytokine, antibody, etc.

As used herein “amelioration” or “treatment” is understood as meaning to lessen or decrease at least one sign, symptom, indication, or effect of a specific disease or condition. For example, amelioration or treatment of retinitis pigmentosa (RP) can be to reduce, delay, or eliminate one or more signs or symptoms of RP including, but not limited to, a reduction in night vision, a reduction in overall visual acuity, a reduction in visual field, a reduction in the cone density in one or more quadrants of the retina, thinning of retina, particularly the outer nuclear layer, reduction in a- or b-wave amplitudes on scotopic or photopic electroretinograms (ERGs); or any other clinically acceptable indicators of disease state or progression. Amelioration and treatment can require the administration of more than one dose of an agent, either alone or in conjunction with other therapeutic agents and interventions. Amelioration or treatment does not require that the disease or condition be cured.

“Antioxidant” as used herein is understood as a molecule capable of slowing or preventing the oxidation of other molecules. Oxidation is a chemical reaction that transfers electrons from a substance to an oxidizing agent. Such reactions can be promoted by or produce superoxide anions or peroxides. Oxidation reactions can produce free radicals, which start chain reactions that damage cells. Antioxidants terminate these chain reactions by removing free radical intermediates, and inhibit other oxidation reactions by being oxidized themselves. As a result, antioxidants are often reducing agents such as thiols, ascorbic acid or polyphenols. Antioxidants include, but are not limited to, α-tocopherol, ascorbic acid, Mn(III)tetrakis (4-benzoic acid) porphyrin, α-lipoic acid, and n-acetylcysteine.

“Co-administration” as used herein is understood as administration of one or more agents to a subject such that the agents are present and active in the subject at the same time. Co-adminsitration does not require a preparation of an admixture of the agents or simultaneous administration of the agents.

The terms “effective amount,” or “effective dose” refers to that amount of an agent to produce the intended pharmacological, therapeutic or preventive result. The pharmacologically effective amount results in the amelioration of one or more signs or symptoms of a disease or condition or the advancement of a disease or condition, or causes the regression of the disease or condition. For example, a therapeutically effective amount preferably refers to the amount of a therapeutic agent that decreases the loss of night vision, the loss of overall visual acuity, the loss of visual field, by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more as compared to an untreated control subject over a defined period of time, e.g., 2 weeks, one month, 2 months, 3 months, 6 months, one year, 2 years, 5 years, or longer. More than one dose may be required to provide an effective dose.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such a treatment may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater liver toxicity.

Thus, in connection with the administration of a drug, a drug which is “effective against” a disease or condition indicates that administration in a clinically appropriate manner results in a beneficial effect for at least a statistically significant fraction of patients, such as a improvement of symptoms, a cure, a reduction in disease signs or symptoms, extension of life, improvement in quality of life, or other effect generally recognized as positive by medical doctors familiar with treating the particular type of disease or condition.

As used herein, the terms “identity” or “percent identity”, refers to the subunit sequence similarity between two polymeric molecules, e.g., two polynucleotides or two polypeptides. When a subunit position in both of the two molecules is occupied by the same monomelic subunit, e.g., if a position in each of two peptides is occupied by serine, then they are identical at that position. The identity between two sequences is a direct function of the number of matching or identical positions, e.g., if half (e.g., 5 positions in a polymer 10 subunits in length), of the positions in two peptide or compound sequences are identical, then the two sequences are 50% identical; if 90% of the positions, e.g., 9 of 10 are matched, the two sequences share 90% sequence identity. The identity between two sequences is a direct function of the number of matching or identical positions. Thus, if a portion of the reference sequence is deleted in a particular peptide, that deleted section is not counted for purposes of calculating sequence identity. Identity is often measured using sequence analysis software e.g., BLASTN or BLASTP (available at (www.ncbi.nih.gov/BLAST). The default parameters for comparing two sequences (e.g., “Blast”-ing two sequences against each other), by BLASTN (for nucleotide sequences) are reward for match=1 , penalty for mismatch=−2, open gap=5, extension gap=2. When using BLASTP for protein sequences, the default parameters are reward for match=0, penalty for mismatch=0, open gap=11, and extension gap=1. Additional, computer programs for determining identity are known in the art.

As used herein, “isolated” or “purified” when used in reference to a polypeptide means that a naturally polypeptide or protein has been removed from its normal physiological environment (e.g., protein isolated from plasma or tissue) or is synthesized in a non-natural environment (e.g., artificially synthesized in an in vitro translation system or using chemical synthesis). Thus, an “isolated” or “purified” polypeptide can be in a cell-free solution or placed in a different cellular environment (e.g., expressed in a heterologous cell type). The term “purified” does not imply that the polypeptide is the only polypeptide present, but that it is essentially free (about 90-95%, up to 99-100% pure) of cellular or organismal material naturally associated with it, and thus is distinguished from naturally occurring polypeptide. Similarly, an isolated nucleic acid is removed from its normal physiological environment. “Isolated” when used in reference to a cell means the cell is in culture (i.e., not in an animal), either cell culture or organ culture, of a primary cell or cell line. Cells can be isolated from a normal animal, a transgenic animal, an animal having spontaneously occurring genetic changes, and/or an animal having a genetic and/or induced disease or condition. An isolated virus or viral vector is a virus that is removed from the cells, typically in culture, in which the virus was produced.

As used herein, “operably linked” is understood as joined, preferably by a covalent linkage, e.g., joining an amino-terminus of one peptide, e.g., expressing an enzyme, to a carboxy terminus of another peptide, e.g., expressing a signal sequence to target the protein to a specific cellular compartment; joining a promoter sequence with a protein coding sequence, in a manner that the two or more components that are operably linked either retain their original activity, or gain an activity upon joining such that the activity of the operably linked portions can be assayed and have detectable activity, e.g., enzymatic activity, protein expression activity. Nucleic acid sequences can also be operably linked in tandem in an expression construct such that both polypeptide encoding sequences are transcribed from a single promoter sequence. Alternatively, each nucleic acid sequence encoding a polypeptide can be operably linked to a single promoter sequence.

“Oxidative stress related ocular disorders” as used herein include, but are not limited to, retinitis pigmentosa, macular degeneration including age related macular degeneration (AMD) both wet and dry, diabetic retinopathy, Lebers optic neuropathy, and optic neuritis.

“Peroxidases” or “a peroxide metabolizing enzyme” are a large family of enzymes that typically catalyze a reaction of the form:

ROOR¹+ electron donor (2 e-)+2H+→ROH+R¹OH For many of these enzymes the optimal substrate is hydrogen peroxide, wherein each R is H, but others are more active with organic hydroperoxides such as lipid peroxides. Peroxidases can contain a heme cofactor in their active sites, or redox -active cysteine or selenocysteine residues.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. For example, pharmaceutically acceptable carriers for administration of cells typically is a carrier acceptable for delivery by injection, and do not include agents such as detergents or other compounds that could damage the cells to be delivered. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations, particularly phosphate buffered saline solutions which are preferred for intraocular delivery.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, intramuscular, intraperotineal, intraocular, intravitreal, subretinal, and/or other routes of parenteral administration. The specific route of administration will depend, inter alia, on the specific cell to be targeted. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

As used herein, “plurality” is understood to mean more than one. For example, a plurality refers to at least two, three, four, five, or more.

A “polypeptide” or “peptide” as used herein is understood as two or more independently selected natural or non-natural amino acids joined by a covalent bond (e.g., a peptide bond). A peptide can include 2, 3, 4, 5, 6, 7, 8, 9, 10, 11, 12, 13, 14, 15, 16, 17, 18, 19, 20, or more natural or non-natural amino acids joined by peptide bonds. Polypeptides as described herein include full length proteins (e.g., fully processed proteins) as well as shorter amino acids sequences (e.g., fragments of naturally occurring proteins or synthetic polypeptide fragments).

As used herein, “prevention” is understood as to limit, reduce the rate or degree of onset, or inhibit the development of at least one sign or symptom of a disease or condition particularly in a subject prone to developing the disease or disorder. For example, a subject having a mutation in a gene, such as the opsin gene, is likely to develop RP. The age of onset of one or more symptoms of the disease can sometimes be determined by the specific mutation. Prevention can include the delay of onset of one or more signs or symptoms of RP and need not be prevention of appearance of at least one sign or symptom of the disease throughout the lifetime of the subject. Prevention can require the administration of more than one dose of an agent or therapeutic.

A “signal sequence” or “signal peptide” as used herein is understood as a peptide sequences that direct proteins into appropriate cellular compartments. Signal sequences are present in proteins that are targeted to specific cellular compartments, or can be added onto proteins that are not targeted to the specific compartment. Signal sequences may or may not be removed from the peptide after translocation into the appropriate cellular compartment. Examples of signal sequences for translocation into or retention in various compartments include, but are not limited to: ER import signal: H₃N-MMSFVSLLLVGILFWATEAEQLTKCEVFQ (SEQ ID NO: 4); ER retention signal: -KDEL-COOH (SEQ ID NO: 5); Mitochondrial import signal: H₃N-MLSLRQSIRFFKPATRTLCSSRYLL-(SEQ ID NO: 6); or H₃N-MLFNLRILLNNAAFRNGHNFMVRNFRCGQPLQLGS (SEQ ID NO: 7); or H₃N-MVLPR LYTATSRAA-(SEQ ID NO: 8); or H₃N-MV[L,A]L[R]P[R,Q,L]R[K] LYT[R,K,I]A[V]T[I]S[R,G,C]RA[V,G]A[V]-(SEQ ID NO: 9) with amino acids listed in [] are acceptable substitutions at the amino acid preceded by the [].

Nuclear import signal: -PPKKKRKV-(SEQ ID NO: 10); Membrane attachment signal sequence: H₃N-GSSKSKPK-(SEQ ID NO: 11). Other mitochondrial signal sequences are known and discussed, for example, in Giazo and Payne, 2003 (Mol. Ther. 7:720-730, incorporated herein by reference).

“Small molecule” as used herein is understood as a compound, typically an organic compound, having a molecular weight of no more than about 1500 Da, 1000 Da, 750 Da, or 500 Da. In an embodiment, a small molecule does not include a polypeptide or nucleic acid including only natural amino acids and/or nucleotides. A “subject” as used herein refers to living organisms. In certain embodiments, the living organism is an animal, hi certain preferred embodiments, the subject is a mammal, hi certain embodiments, the subject is a domesticated mammal or a primate including a non-human primate. Examples of subjects include humans, monkeys, dogs, cats, mice, rats, cows, horses, goats, and sheep. A human subject may also be referred to as a patient.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions such as RP and age-related macular degeneration (AMD) is within the ability of those in the art. Subjects suffering from, and suspected of suffering from, a specific disease, condition, or syndrome are not necessarily two distinct groups.

As used herein, “superoxide dismutase” is understood as an enzyme that dismutation of superoxide into oxygen and hydrogen peroxide. Examples include, but are not limited to SOD1, SOD2, and SOD3. SOD1 and SOD3 are two isoforms of Cu—Zn-containing superoxide dismutase enzymes exist in mammals. Cu—Zn—SOD or SOD1, is found in the intracellular space, and extracellular SOD (ECSOD or SOD3) predominantly is found in the extracellular matrix of most tissues.

“Therapeutically effective amount,” as used herein refers to an amount of an agent which is effective, upon single or multiple dose administration to the cell or subject, in prolonging the survivability of the patient with such a disorder, reducing one or more signs or symptoms of the disorder, preventing or delaying and the like beyond that expected in the absence of such treatment.

An agent or other therapeutic intervention can be administered to a subject, either alone or in combination with one or more additional therapeutic agents or interventions, as a pharmaceutical composition in mixture with conventional excipient, e.g., pharmaceutically acceptable carrier, or therapeutic treatments.

The pharmaceutical agents may be conveniently administered in unit dosage form and may be prepared by any of the methods well known in the pharmaceutical arts, e.g., as described in Remington's Pharmaceutical Sciences (Mack Pub. Co., Easton, Pa., 1985). Formulations for parenteral administration may contain as common excipients such as sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of certain agents.

It will be appreciated that the actual preferred amounts of active compounds used in a given therapy will vary according to e.g., the specific compound being utilized, the particular composition formulated, the mode of administration and characteristics of the subject, e.g., the species, sex, weight, general health and age of the subject. Optimal administration rates for a given protocol of administration can be readily ascertained by those skilled in the art using conventional dosage determination tests conducted with regard to the foregoing guidelines.

As used herein, “susceptible to” or “prone to” or “predisposed to” a specific disease or condition and the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

Ranges provided herein are understood to be shorthand for all of the values within the range.

As used herein, the embodiments of this invention are defined to include pharmaceutically acceptable derivatives thereof A “pharmaceutically acceptable derivative” means any pharmaceutically acceptable salt, ester, salt of an ester, or other derivative of a compound of this invention which, upon administration to a recipient, is capable of providing (directly or indirectly) a compound of this invention. Particularly favored derivatives are those that increase the bioavailability of the compounds of this invention when such compounds are administered to a mammal (e.g., by allowing an orally administered compound to be more readily absorbed into the blood, to increase serum stability or decrease clearance rate of the compound) or which enhance delivery of the parent compound to a biological compartment (e.g., the brain or lymphatic system) relative to the parent species. Derivatives include derivatives where a group which enhances aqueous solubility or active transport through the gut membrane is appended to the structure of formulae described herein.

The embodiments of this invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion. Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g. , sodium), alkaline earth metal (e.g., magnesium), ammonium and N-(alkyl)₄₊ salts. This invention also envisions the quaternization of any basic nitrogen-containing groups of the compounds disclosed herein. Water or oil-soluble or dispersible products may be obtained by such quaternization.

The embodiments of the invention can, for example, be administered by injection, intraocularly, intravitreally, subretinal, intravenously, intraarterially, subdermally, intraperitoneally, intramuscularly, or subcutaneously; or orally, buccally, nasally, transmucosally, directly to a diseases organ by catheter, topically, or in an ophthalmic preparation, with a dosage ranging from about 0.001 to about 100 mg/kg of body weight, or according to the requirements of the particular drug and more preferably from 0.5-10 mg/kg of body weight. It is understood that when a compound is delivered directly to the eye, considerations such as body weight have less bearing on the dose. For ocular administration, especially subretinal administration, the total volume for administration is of substantial concern with the preferred dosage being in the smallest volume possible for dosing. For administration of viral particles, dosages are typically provided by number of virus particles (or viral genomes) and effective dosages would range from about 10³ to 10¹² particles, 10⁵ to 10″ particles, 10⁶ to 10¹⁰ particles, 10⁸ to 10¹¹ particles, or 10⁹ to 10¹⁰ particles. The effective dose can be the number of particles delivered for each expression construct to be delivered when different expression constructs encoding different genes are administered separately. In alternative embodiment, the effective dose can be the total number of particles administered, of one or more types. The methods herein contemplate administration of an effective amount of compound or compound composition to achieve the desired or stated effect.

In accordance with one or more embodiments, the present invention provides for the use of the compositions described herein for the prevention, amelioration, or treatment of a disease or condition associated with oxidative stress in a subject, comprising administration of the composition to the subject in an amount effective to increase the amount of glutathione expressed in the tissues of the subject.

Frequency of dosing will depend on the agent administered, the progression of the disease or condition in the subject, and other considerations known to those of skill in the art. For example, pharmacokinetic and pharmacodynamic considerations for compositions delivered to the eye, or even compartments within the eye, are different, e.g., clearance in the subretinal space is very low. Therefore, dosing can be as infrequent as once a month, once every three months, once every six months, once a year, once every five years, or less. If systemic administration of antioxidants is to be performed in conjunction with administration of expression constructs to the subretinal space, it is expected that the dosing frequency of the antioxidant will be higher than the expression construct, e.g., one or more times daily, one or more times weekly.

Dosing may be determined in conjunction with monitoring of one or more signs or symptoms of the disease, e.g., visual acuity, visual field, night visions, etc. The amount of active ingredient that may be combined with the carrier materials to produce a single dosage form will vary depending upon the host treated and the particular mode of administration. A typical preparation will contain from about 1% to about 95% active compound (w/w). Alternatively, such preparations contain from about 20% to about 80% active compound. Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

The term “pharmaceutically acceptable carrier” refers to a carrier that can be administered to a patient, together with an embodiment of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-a. -tocopherol polyethyleneglycol 1000 succinate, surfactants used in pharmaceutical dosage forms such as Tween® or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropyle-ne-block polymers, polyethylene glycol and wool fat. Cyclodextrins such as alpha-, beta-, and gamma-cyclodextrin, may also be advantageously used to enhance delivery of embodiments described herein.

The pharmaceutical compositions of this invention may be administered enterally for example by oral administration, parenterally, intraocularly, by inhalation spray, topically, nasally, buccally, or via an implanted reservoir, preferably by oral or vaginal administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases, or buffers to enhance the stability of the formulated compound or its delivery form. The term parenteral as used herein includes intraocular, subcutaneous, intracutaneous, intravenous, intramuscular, intraarticular, intraarterial, intrasynovial, intrastemal, intrathecal, intralesional, and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, TWEEN® 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as TWEENs® or SPANs® and/or other similar emulsifying agents or bioavailability enhancers which are commonly used in the manufacture of pharmaceutically acceptable solid, liquid, or other dosage forms may also be used for the purposes of formulation.

When the compositions of this invention comprise a combination of a nucleic acids and expression constructs described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition. Effective dosages of the expression constructs of the invention to be administered may be determined through procedures well known to those in the art which address such parameters as biological half-life, bioavailability, and toxicity.

Gene delivery compositions and methods for gene delivery to various organs and cell types in the body are known to those of skill in the art. Such compositions and methods are provided, for example in U.S. Pat. Nos. 7,459,153; 7,041,284; 6,849,454; 6,410,011; 6,027,721; and 5,705,151, all of which are incorporated herein by reference. Expression constructs provided in the listed patents and any other known expression constructs for gene delivery can be used in the compositions and methods of the invention.

Gene Delivery to the Eye

The eye has unique advantages as a target organ for the development of novel therapies and is often regarded as a valuable model system for gene therapy. It is a relatively small target organ with highly compartmentalized anatomy in which it is possible to deliver small volumes of expression vectors for gene delivery, in the context of a viral particle, as nucleic acid alone, or nucleic acid complexed with other agents. It is possible to obtain precise, efficient, and stable transduction of a variety of ocular tissues with attenuated immune responses due to the immune privileged nature of the eye. The risks of systemic side effects for eye procedures are minimal Further, if only one eye is treated, the untreated eye may serve as a useful control. Gene therapy offers a potentially powerful modality for the management of both rare and common complex acquired disorders (Gene Therapy 15:633-634, 2008).

Compositions and methods provided herein include the use of gene delivery to the eye for expression of human glutamate cysteine ligase, human glutathione synthase, or both. In three stage I clinical trials for the treatment of ocular disease, specifically Leber Congenital Amaurosis, an incurable retinal degeneration that causes severe vision loss, gene delivery using an adenoassociated virus administered subretinally was demonstrated to be safe. Moreover, as a secondary outcome, improvement in visual function was observed in seven of the first nine treated patients. (N Engl J Med. 358:2231-9 2008; N Engl J Med. 358:2240-8 2008; N Engl J Med. 358:2282-4 2008; Hum Gene Ther. Sep 7; [Epub ahead of print 2008]). These data demonstrate that gene delivery can be effective for the treatment of an otherwise incurable ocular disease.

The viral vectors used in each of the studies demonstrate that various gene therapy viral vector designs can be useful for gene deliver. Methods of viral vector design and generation are well known to those of skill in the art, and methods of preparation of viral vectors can be performed by any of a number of companies as demonstrated below. Expression constructs provided herein can be inserted into any of the exemplary viral vectors listed below. Alternatively, viral vectors can be generated base on the examples provided below.

For example, in the Bainbridge study, the tgAAG76 vector, a recombinant adeno-associated virus vector of serotype 2 was used for gene delivery. The vector contains the human RPE65 coding sequence driven by a 1400-bp fragment of the human RPE65 promoter and terminated by the bovine growth hormone polyadenylation site, as described elsewhere. The vector was produced by Targeted Genetics Corporation according to Good Manufacturing Practice guidelines with the use of a B50 packaging cell line, an adenovirus-adeno-associated virus hybrid shuttle vector containing the tgAAG76 vector genome, and an adenovirus 5 helper virus. The vector was filled in a buffered saline solution at a titer of 1x10¹¹ vector particles per milliliter and frozen in 1-ml aliquots at −70° C.

Further AAV vectors are provided in the review by Rolling (Gene Therapy 11:S26-S32, 2004). Hybrid AAV viral vectors, including AAV 2/4 and AAV2/5 vectors are provided, for example, by US Patent 7,172,893. Such hybrid virus particles include a parvovirus capsid and a nucleic acid having at least one adeno-associated virus (AAV) serotype 2 inverted terminal repeat packaged in the parvovirus capsid. However, the serotypes of the AAV capsid and said at least one of the AAV inverted terminal repeat are different. For example, a hybrid AAV2/5 virus in which a recombinant AAV2 genome (with AAV2 ITRs) is packaged within an AAV Type 5 capsid.

Self-complementary AAV (scAAV) vectors have been developed to circumvent rate-limiting second-strand synthesis in single-stranded AAV vector genomes and to facilitate robust transgene expression at a minimal dose (IOVS: 48:3324-3328, 2007). Self-complementary AAV-vectors were demonstrated to provide almost immediate and robust expression of the reporter gene inserted in the vector. Subretinal injection of 5×10 viral particles (vp) of scAAV. CMV-GFP resulted in green fluorescent protein (GFP) expression in almost all retinal pigment epithelial (RPE) cells within the area of the small detachment caused by the injection by 3 days and strong, diffuse expression by 7 days. Expression was strong in all retinal cell layers by days 14 and 28. In contrast, 3 days after subretinal injection of 5×10 vp of ssAAV. CMV-GFP, GFP expression was detectable in few RPE cells. Moreover, the ssAAV vector required 14 days for the attainment of expression levels comparable to those observed using scAAV at day 3. Expression in photoreceptors was not detectable until day 28 using the ssAAV vector. The use of the scAAV vector in the gene delivery methods of the invention can allow for prompt and robust expression from the expression construct. Moreover, the higher level of expression from the scAAV viral vectors can allow for delivery to of the viral particles intravitreally rather than subretinally. Various recombinant AAV viral vectors have been designed including one or more mutations in capsid proteins or other viral proteins. It is understood that the use of such modified AAV viral vectors falls within the scope of the instant invention. It is contemplated that self-complementary adeno-associated vectors can be used in the compositions of the present invention. Under normal circumstances, AAV packages a single-stranded DNA molecule of up to 4800 nucleotides in length. Following infection of cells by the virus, the intrinsic molecular machinery of the cell is required for conversion of single-stranded DNA into double stranded form. The double-stranded form is then capable of being transcribed, thereby allowing expression of the delivered gene to commence. It has been shown in a number of cell and tissue types that second strand synthesis of DNA by the host cell is the rate-limiting step in expression. By virtue of already being packaged as a double stranded DNA molecule, self-complementary AAV (scAAV) bypasses this step, thereby greatly reducing the time to onset of gene expression.

Self-complementary AAV is generated through the use of vector plasmid with a mutation in one of the terminal resolution sequences of the AAV virus. This mutation leads to the packaging of a self-complementary, double-stranded DNA molecule covalently linked at one end. Vector genomes are required to be approximately half genome size (2.4 KB) in order to package effectively in the normal AAV capsid. Because of this size limitation, large promoters are unsuitable for use with scAAV. Most broad applications to date have used the cytomegalovirus immediate early promoter (CMV) alone for driving transgene expression. However, it has been shown by others that transgene expression with CMV markedly drops off in certain tissue types, such as eye and liver, sometimes as early as two weeks post-injection. A long acting, ubiquitous promoter of small size would be very useful in a scAAV system.

Nucleic Acid Regulatory Sequences

The invention provides expression constructs that include any regulatory sequences that are functional in the cells in which protein expression is desired, e.g., retinal pigment epithelial (RPE) cells, rod cells, cone cells, etc. For example, cell and tissue specific promoters such as the interphotoreceptor retinoid binding protein (J. Biochem. 125:1189-1199, (1999), BBRC, 181:159-165, 1991;), cone arrestin promoter (IOVS. 45:3877-3884, 2004), RPE65 promoter, and cis-Retinaldehyde-binding protein (CRALBP) promoter is a retinal-pigment-epithelium (RPE)-specific promoter (2,265 bp) when administered subretinally in a rAAV vector can be used in the expression constructs of the instant invention. Alternatively, non-tissue specific promoters including viral promoters such as cytomegalovirus (CMV) promoter and β-actin promoter can be used such as the chicken β-actin (CBA) promoter.

The chimeric CMV-chicken [beta]-actin promoter (CBA) has been utilized extensively as a promoter that supports expression in a wide variety of cells when in rAAV vectors delivered to retina, including in the clinical trials discussed herein. In addition to broad tropism, the present inventors have observed that CBA also has the capacity to promote expression for long periods post infection (Mol. Ther., 2005, 12:1072-1082). CBA is −1700 base pairs in length, too large in most cases to be used in conjunction with scAAV to deliver cDNAs (over 300 bps pairs in length). CBA is a ubiquitous strong promoter composed of a cytomegalovirus (CMV) immediate-early enhancer (381 bp) and a

CBA promoter-exonl-intronl element (1,352 bp) (Proc Natl Acad Sci., USA. 2002 Jun. 25; 99(13): 8909-8914). A shortened CBA promoter sequence, the smCBA promoter sequence, has also been described in which the The total size of smCBA is 953 bps versus 1714 bps for full length CBA. The smCBA promoter is described in (Hum. Gene Ther. 14: 143-152, 2003) and (IOVS, 2006, 47:3745-3753).

Other regulatory sequences for inclusion in expression constructs include poly-A signal sequences, for example SV40 polyA signal sequences. The inclusion of a splice site (i.e., exon flanked by two introns) has been demonstrated to be useful to increase gene expression of proteins from expression constructs. For viral sequences, the use of viral sequences including inverted terminal repeats, for example in AAV viral vectors can be useful. Certain viral genes can also be useful to assist the virus in evading the immune response after administration to the subject.

In certain embodiments of the invention, the viral vectors used are replication deficient, but contain some of the viral coding sequences to allow for replication of the virus in appropriate cell lines. The specific viral genes to be partially or fully deleted from the viral coding sequence is a matter of choice, as is the specific cell line in which the virus is propagated. Such considerations are well known to those of skill in the art. Peptide signal sequences hi order for proteins, either endogenously or heterologously expressed, to function properly must exist in the appropriate compartment of the cell.

Codon optimization Expression construct design and generation can include the use of codon optimization. The degeneracy of the genetic code is well known with more than one nucleotide triplet coding for most of the amino acids, e.g., each leucine, arginine, and serine are encoded by five different codons each. It is possible to design multiple nucleotide sequences that encode a single amino acid sequence. Redesign of a nucleotide sequence without changing the sequence of the polypeptide encoded is well within the ability of those of skill in the art.

### EXAMPLES

Cell Culture. Transfected and control cells were grown in Dulbecco's Modified Eagles's Medium (DMEM) supplemented with 10% fetal bovine serum (FBS), 100 U/ml penicillin and 100 pg/ml streptomycin (all from Invitrogen Corp, Carlsbad, Calif.) at 37° C. and 5% CO₂. Confluent cells were washed and placed in growth medium supplemented with or without 7 mM paraquat (Aldrich, Wilwaukee, Wis.), or 0.5 mM H₂O₂ (Sigma, St. Louis, Mo.) for one day. To expose cells to hyperoxia, cells were grown to confluence in a 25 cm flask, which was filled with 100% oxygen for 1 minute, then the cap was loosened and the flask was returned to the 5% CO₂ incubator. This was repeated twice a day until the cells were scraped into lysis buffer and collected as described previously (Lu, 2006. J Cell Physiol 206: 119-125, incorporated herein by reference).

Assessment of Superoxide Radicals with Hydroethidine.

As previously described (Free Radic Biol Med 45: 905-912 2008; and Science 318: 1645-1647, 2008) in situ production of superoxide radicals was evaluated using hydroethidine, which in the presence of superoxide radicals is converted to ethidium, which binds DNA and emits red fluorescence at 600 nm. Briefly, mice were given two 20-mg/kg intraperitoneal injections 30 minutes apart of freshly prepared hydroethidine (Invitrogen, Carlsbad, Calif.) and euthanized 18 hours after injection. Eyes were rapidly removed and 10-μm frozen sections were fixed in 4% paraformaldehyde for 20 minutes at room temperature, rinsed with phosphate-buffered saline (PBS), and counterstained for 5 minutes at room temperature with the nuclear dye Hoechst 33258 (1 :10,000; Sigma, St Louis, Mo.). After rinsing in PBS, slides were mounted with Aquamount solution and evaluated for fluorescence (excitation: 543 nm, emission>590 nm) with a LSM 510 META confocal microscope. Images were captured using the same exposure time for each section. ELISA for protein carbonyl content. Retinas were homogenized in lysis buffer and centrifuged at 16,000g for 5 minutes at 4° C. and the protein concentration of the supernatant was measured using a Bio-Rad Protein Assay Kit (Bio-Rad). Samples were adjusted to 4 mg/ml by dilution with Trisbuffered saline, and protein carbonyl content was determined by ELISA, as previously described (Proc Natil Acad Sci USA 103 : 11300-11305 2006; Antioxid Redox Signal, epub ahead of print 2008).

Measurement of Cone Cell Density.

Cone density was measured as previously described (Proc Natil Acad Sci USA 103:11300-11305, 2006). Briefly, each mouse was euthanized, and eyes were carefully removed and were fixed in 4% paraformaldehyde for 3 hours or overnight at 4° C. After washing with PBS, the cornea, iris, and lens were removed. A small triangle cut was made at 12:00 in the retina for future orientation and after four cuts equidistant around the circumference, the entire retina was carefully dissected from the eye cup and any adherent retinal pigmented epithelium was removed. Retinas were placed in 10% normal goat serum in PBS for 30 minutes at room temperature, incubated for 1 hour at room temperature in 1:100 rhodamine-conjugated peanut agglutinin (Vector Laboratories, Burlingame, Calif.) in PBS containing 1% normal goat serum, and flat mounted. The retinas were examined with a Zeiss LSM 510 META confocal microscope (Carl Zeiss, Oberkochen, Germany) with a Zeiss Plan-Apochromat 20×/0.75 NA objective using an excitation wavelength of 543 ran to detect rhodamine fluorescence. Images were acquired in the frame scan mode. The number of cones was determined by image analysis within four 230 mm*230 mm squares located 1 mm (experimental mice) or 0.5 mm control mice) superior, inferior, temporal, and nasal to the center of the optic nerve. The investigator was masked with respect to experimental group.

Recording of ERGs.

An Espion ERG Diagnosys machine (DiagnoSYS LLL, Littleton, Mass.) was used to record ERGs as previously described (Proc Natil Acad Sci USA 103: 11300-11305 2006; J Cell Physiol 213: 809-815 2007; J Neurosci 23: 4164-4172 2003; J Cell Physiol 217: 13-22, 2008). For scotopic recordings, mice were adapted to dark overnight, and for photopic recordings, mice were adapted to background white light at an intensity of 30 cd/m² for 10 minutes. The mice were anesthetized with an intraperitoneal injection of ketamine hydrochloride (100 mg/kg body weight) and xylazine (5 mg/kg body weight). Pupils were dilated with Midrin P containing of 0.5% tropicamide and 0.5% phenylephrine, hydrochloride (Santen Pharmaceutical, Osaka, Japan). The mice were placed on a pad heated to 39° C. and platinum loop electrodes were placed on each cornea after application of Gonioscopic prism solution (Alcon Labs, Fort Worth, Tex.). A reference electrode was placed subcutaneously in the anterior scalp between the eyes and a ground electrode was inserted into the tail. The head of the mouse was held in a standardized position in a ganzfeld bowl illuminator that ensured equal illumination of the eyes. Recordings for both eyes were made simultaneously with electrical impedance balanced. Scotopic ERGs were recorded at six intensity levels of white light ranging from −3.00 to 1.40 log cd-s/m². Six measurements were averaged at each flash intensity. Low background photopic ERGs were recorded at 1.48 log cd-s/m² under a background of 10 cd/m². Sixty photopic measurements were taken and the average value was recorded.

Statistical Analysis.

Statistical comparisons were done using ANOVA with Dunnett's test for multiple comparisons, or by using Tukey-Kramer's test for multiple comparisons and unpaired Student's t-test or Welch's t-test for two comparisons, as noted. Differences were judged statistically significant at P<0.05 or P<0.01, as noted.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1.-4. (canceled)

5. An expression construct for expression of human glutamate cysteine ligase and human glutathione synthase comprising a polynucleotide encoded by the the nucleotide sequences of SEQ ID NOS: 1-3, or functional portions or fragments thereof.

6. The expression construct of claim 5, wherein the expression construct is included in a viral vector selected from the group consisting of an adenoviral (Ad) vector, an adeno-associated viral vector (AAV), a lentiviral vector, and a herpes simplex viral (HSV) vector.

7. The expression construct of claim 6, wherein the AAV viral vector is selected from the group consisting of AAV2 viral vectors, hybrid AAV2/4 viral vectors, and hybrid AAV2/5 viral vectors.

8. The expression construct of claim 7, wherein nucleic acid in the AAV viral vector is self-complementary.

9. The expression construct of claim 5, wherein the viral vector is replication competent.

10. The expression construct of claim 5, wherein the viral vector is replication incompetent.

11. The expression construct of claim 5, wherein the coding sequences for expression of the human glutamate cysteine ligase and human glutathione synthase enzymes are incorporated into a single expression vector.

12. The expression construct of claim 5, wherein the coding sequences for expression of the human glutamate cysteine ligase and human glutathione synthase enzymes are incorporated into separate expression vectors.

13. The expression construct of claim 5, wherein the expression construct comprises a promoter sequence selected from the group consisting of an interphotoreceptor retinoid-binding protein (IRBP) promoter, a cytomegalovirus (CMV) promoter, a α-globin promoter, cone arrestin promoter, RPE65 promoter, cis-Retinaldehyde-binding protein (CRALBP) promoter is a retinal-pigment-epithelium (RPE)-specific promoter, chicken β-actin (CBA) promoter, and small chicken β-actin (smCBA) promoter.

14. The expression construct of claim 5, wherein the human glutamate cysteine ligase and human glutathione synthase enzymes or both are independently operably linked to a signal sequence selected from the group consisting of mitochondrial signal sequence, endoplasmic reticulum signal sequence, and nuclear signal sequence.

15. A method for treatment of a disease or condition associated with oxidative stress in a subject, comprising administration of the expression construct of claim 5 to the subject in an amount effective to increase the amount of glutathione expressed in the tissues of the subject.

16. The method of claim 15, wherein administration comprises administration by a route selected from the group consisting of intravitreally, subretinal, intravenously, intraarterially, subdermally, intraperitoneally, intramuscularly, subcutaneously, orally, buccally, nasally, transmucosally, directly to a diseases organ by catheter, and topically.

17. The method of claim 16, wherein the composition is administered to the eye by a route selected from the group consisting of subretinal and intravitreal.

18. The method of claim 15, further comprising administration of an antioxidant to the subject.

19. The method of claim 15, wherein the disease or condition associated with oxidative stress in an eye is selected from the group consisting of atherosclerosis, Parkinson's disease, heart failure, myocardial infarction, Alzheimer's disease, diabetes, chronic lung disease, diseases associated with mitochondrial dysfunction, diseases associated with chronic inflammation, retinitis pigmentosa, wet age related macular degeneration, dry age related macular degeneration, diabetic retinopathy, Lebers optic neuropathy, and optic neuritis.

20. The method of 19, wherein a disease or condition associated with oxidative stress comprises an ocular disease or condition associated with oxidative stress.


SEQUENCE LISTING (14 sequences)

SEQ ID NO: 1 - DNA, 825 bp, Homo sapiens
atgggcaccgacagccgcgcggccaaggcgctcctggcgcgggcccgcaccctgcacctgcagacggggaacctgctgaactggggccgcctgcggaagaagtgcccgtccacgcacagcgaggagcttcatgattgtatccaaaaaaccttgaatgaatggagttcccaaatcaacccagatttggtcagggagtttccagatgtcttggaatgcactgtatctcatgcagtagaaaagataaatcctgatgaaagagaagaaatgaaagtttctgcaaaactgttcattgtagaatcaaactcttcatcatcaactagaagtgcagttgacatggcctgttcagtccttggagttgcacagctggattctgtgatcattgcttcacctcctattgaagatggagttaatctttccttggagcatttacagccttactgggaggaattagaaaacttagttcagagcaaaaagattgttgccataggtacctctgatctagacaaaacacagttggaacagctgtatcagtgggcacaggtaaaaccaaatagtaaccaagttaatcttgcctcctgctgtgtgatgccaccagatttgactgcatttgctaaacaatttgacatacagctgttgactcacaatgatccaaaagaactgctttctgaagcaagtttccaagaagctcttcaggaaagcattcctgacattcaagcgcacgagtgggtgccgctgtggctactgcggtattcggtcattgtgaaaagtagaggaattatcaaatcaaaaggctacattttacaagctaaaagaaggggttcttaa

SEQ ID NO: 2 - DNA, 1914 bp, Homo sapiens
atggggctgctgtcccagggctcgccgctgagctgggaggaaaccaagcgccatgccgaccacgtgcggcggcacgggatcctccagttcctgcacatctaccacgccgtcaaggaccggcacaaggacgttctcaagtggggcgatgaggtggaatacatgttggtatcttttgatcatgaaaataaaaaagtccggttggtcctgtctggggagaaagttcttgaaactctgcaagagaagggggaaaggacaaacccaaaccatcctaccctttggagaccagagtatgggagttacatgattgaagggacaccaggacagccctacggaggaacaatgtccgagttcaatacagttgaggccaacatgcgaaaacgccggaaggaggctacttctatattagaagaaaatcaggctctttgcacaataacttcatttcccagattaggctgtcctgggttcacactgcccgaggtcaaacccaacccagtggaaggaggagcttccaagtccctcttctttccagatgaagcaataaacaagcaccctcgcttcagtaccttaacaagaaatatccgacataggagaggagaaaaggttgtcatcaatgtaccaatatttaaggacaagaatacaccatctccatttatagaaacatttactgaggatgatgaagcttcaagggcttctaagccggatcatatttacatggatgccatgggatttggaatgggcaattgctgtctccaggtgacattccaagcctgcagtatatctgaggccagatacctttatgatcagttggctactatctgtccaattgttatggctttgagtgctgcatctcccttttaccgaggctatgtgtcagacattgattgtcgctggggagtgatttctgcatctgtagatgatagaactcgggaggagcgaggactggagccattgaagaacaataactataggatcagtaaatcccgatatgactcaatagacagctatttatctaagtgtggtgagaaatataatgacatcgacttgacgatagataaagagatctacgaacagctgttgcaggaaggcattgatcatctcctggcccagcatgttgctcatctctttattagagacccactgacactgtttgaagagaaaatacacctggatgatgctaatgagtctgaccattttgagaatattcagtccacaaattggcagacaatgagatttaagccccctcctccaaactcagacattggatggagagtagaatttcgacccatggaggtgcaattaacagactttgagaactctgcctatgtggtgtttgtggtactgctcaccagagtgatcctttcctacaaattggattttctcattccactgtcaaaggttgatgagaacatgaaggtagcacagaaaagagatgctgtcttgcagggaatgttttatttcaggaaagatatttgcaaaggtggcaatgcagtggtggatggttgtggcaaggcccagaacagcacggagctcgctgcagaggagtacaccctcatgagcatagacaccatcatcaatgggaaggaaggtgtgtttcctggactgatcccaattctgaactcttaccttgaaaacatggaagtggatgtggacaccagatgtagtattctgaactacctaaagctaattaagaagagagcatctggagaactaatgacagttgccagatggatgagggagtttatcgcaaaccatcctgactacaagcaagacagtgtcataactgatgaaatgaattatagccttattttgaagtgtaaccaaattgcaaatgaattatgtgaatgcccagagttacttggatcagcatttaggaaagtaaaatatagtggaagtaaaactgactcatccaactag

SEQ ID NO: 3 - DNA, 1425 bp, Homo sapiens
atggccaccaactgggggagcctcttgcaggataaacagcagctagaggagctggcacggcaggccgtggaccgggccctggctgagggagtattgctgaggacctcacaggagcccacttcctcggaggtggtgagctatgccccattcacgctcttcccctcactggtccccagtgccctgctggagcaagcctatgctgtgcagatggacttcaacctgctagtggatgctgtcagccagaacgctgccttcctggagcaaactctttccagcaccatcaaacaggatgactttaccgctcgtctctttgacatccacaagcaagtcctaaaagagggcattgcccagactgtgttcctgggcctgaatcgctcagactacatgttccagcgcagcgcagatggctccccagccctgaaacagatcgaaatcaacaccatctctgccagctttgggggcctggcctcccggaccccagctgtgcaccgacatgttctcagtgtcctgagtaagaccaaagaagctggcaagatcctctctaataatcccagcaagggactggccctgggaattgccaaagcctgggagctctacggctcacccaatgctctggtgctactgattgctcaagagaaggaaagaaacatatttgaccagcgtgccatagagaatgagctactggccaggaacatccatgtgatccgacgaacatttgaagatatctctgaaaaggggtctctggaccaagaccgaaggctgtttgtggatggccaggaaattgctgtggtttacttccgggatggctacatgcctcgtcagtacagtctacagaattgggaagcacgtctactgctggagaggtcacatgctgccaagtgcccagacattgccacccagctggctgggactaagaaggtgcagcaggagctaagcaggccgggcatgctggagatgttgctccctggccagcctgaggctgtggcccgcctccgcgccacctttgctggcctctactcactggatgtgggtgaagaaggggaccaggccatcgccgaggcccttgctgcccctagccggtttgtgctaaagccccagagagagggtggaggtaacaacctatatggggaggaaatggtacaggccctgaaacagctgaaggacagtgaggagagggcctcctacatcctcatggagaagatcgaacctgagccttttgagaattgcctgctacggcctggcagccctgcccgagtggtccagtgcatttcagagctgggcatctttggggtctatgtcaggcaggaaaagacactcgtgatgaacaagcacgtggggcatctacttcgaaccaaagccatcgagcatgcagatggtggtgtggcagcgggagtggcagtcctggacaacccataccctgtgtga

SEQ ID NO: 4 - protein, 29 aa, Homo sapiens
MMSFVSLLLVGILFWATEAEQLTKCEVFQ

SEQ ID NO: 5 - protein, 4 aa, Homo sapiens
KDEL

SEQ ID NO: 6 - protein, 25 aa, Homo sapiens
MLSLRQSIRFFKPATRTLCSSRYLL

SEQ ID NO: 7 - protein, 35 aa, Homo sapiens
MLFNLRILLNNAAFRNGHNFMVRNFRCGQPLQLGS

SEQ ID NO: 8 - protein, 14 aa, Homo sapiens
MVLPRLYTATSRAA

SEQ ID NO: 9 - protein, 14 aa, Homo sapiens
MVLPRLYTATSRAA

SEQ ID NO: 10 - protein, 8 aa, Homo sapiens
PPKKKRKV

SEQ ID NO: 11 - protein, 8 aa, Homo sapiens
GSSKSKPK

SEQ ID NO: 12 - protein, 274 aa, Homo sapiens
MGTDSRAAKALLARARTLHLQTGNLLNWGRLRKKCPSTHSEELHDCIQKTLNEWSSQINPDLVREFPDVLECTVSHAVEKINPDEREEMKVSAKLFIVESNSSSSTRSAVDMACSVLGVAQLDSVIIASPPIEDGVNLSLEHLQPYWEELENLVQSKKIVAIGTSDLDKTQLEQLYQWAQVKPNSNQVNLASCCVMPPDLTAFAKQFDIQLLTHNDPKELLSEASFQEALQESIPDIQAHEWVPLWLLRYSVIVKSRGIIKSKGYILQAKRRGS

SEQ ID NO: 13 - protein, 637 aa, Homo sapiens
MGLLSQGSPLSWEETKRHADHVRRHGILQFLHIYHAVKDRHKDVLKWGDEVEYMLVSFDHENKKVRLVLSGEKVLETLQEKGERTNPNHPTLWRPEYGSYMIEGTPGQPYGGTMSEFNTVEANMRKRRKEATSILEENQALCTITSFPRLGCPGFTLPEVKPNPVEGGASKSLFFPDEAINKHPRFSTLTRNIRHRRGEKVVINVPIFKDKNTPSPFIETFTEDDEASRASKPDHIYMDAMGFGMGNCCLQVTFQACSISEARYLYDQLATICPIVMALSAASPFYRGYVSDIDCRWGVISASVDDRTREERGLEPLKNNNYRISKSRYDSIDSYLSKCGEKYNDIDLTIDKEIYEQLLQEGIDHLLAQHVAHLFIRDPLTLFEEKIHLDDANESDHFENIQSTNWQTMRFKPPPPNSDIGWRVEFRPMEVQLTDFENSAYVVFVVLLTRVILSYKLDFLIPLSKVDENMKVAQKRDAVLQGMFYFRKDICKGGNAVVDGCGKAQNSTELAAEEYTLMSIDTIINGKEGVFPGLIPILNSYLENMEVDVDTRCSILNYLKLIKKRASGELMTVARWMREFIANHPDYKQDSVITDEMNYSLILKCNQIANELCECPELLGSAFRKVKYSGSKTDSSN

SEQ ID NO: 14 - protein, 474 aa, Homo sapiens
MATNWGSLLQDKQQLEELARQAVDRALAEGVLLRTSQEPTSSEVVSYAPFTLFPSLVPSALLEQAYAVQMDFNLLVDAVSQNAAFLEQTLSSTIKQDDFTARLFDIHKQVLKEGIAQTVFLGLNRSDYMFQRSADGSPALKQIEINTISASFGGLASRTPAVHRHVLSVLSKTKEAGKILSNNPSKGLALGIAKAWELYGSPNALVLLIAQEKERNIFDQRAIENELLARNIHVIRRTFEDISEKGSLDQDRRLFVDGQEIAVVYFRDGYMPRQYSLQNWEARLLLERSHAAKCPDIATQLAGTKKVQQELSRPGMLEMLLPGQPEAVARLRATFAGLYSLDVGEEGDQAIAEALAAPSRFVLKPQREGGGNNLYGEEMVQALKQLKDSEERASYILMEKIEPEPFENCLLRPGSPARVVQCISELGIFGVYVRQEKTLVMNKHVGHLLRTKAIEHADGGVAAGVAVLDNPYPV